(19) United States
(12) Patent Application Publication
(10) Pub. No.: US 2007/0010488 A1
(43) Pub. Date: Jan. 11, 2007

(54) Compounds for modulating cell proliferation

(76) Inventors: Khairia Youssef, Riyadh (SA); Magda A. El-Sherbeny, Mansoura (EG)

Correspondence Address: FISH & NEAVE IP GROUP;ROPES & GRAY LLP, ONE INTERNATIONAL PLACE, BOSTON, MA, 02110-2624, US

(21) Appl. No.: 11/404,511

(22) Filed: Apr. 13, 2006

Related U.S. Application Data
(60) Provisional application No. 60/670,844, filed on Apr. 13, 2005.

(51) Int. Cl. A61K 31/695; A61K 31/445; A61K 31/35

(52) U.S. Cl. 514/63; 514/317; 514/460

(57) ABSTRACT

Compounds that modulate cell proliferation are taught. These compounds are useful as inhibitors of abnormal cell proliferation.

### CROSS-REFERENCE TO RELATED APPLICATIONS

This application claims the benefit of U.S. Provisional Patent Application No. 60/670,844, filed Apr. 13, 2005, the specification of which is hereby incorporated by reference in its entirety.

### FIELD OF THE INVENTION

This invention relates to novel compounds which are useful for treating a variety of cell proliferative disorders such as cancer.

### BACKGROUND OF THE INVENTION

A wide range of growth factors coordinate cell proliferation and differentiation. Malignant cells arise as a result of a stepwise progression of events that include the unregulated expression of growth factors or components of their signaling pathways. Tyrosine phosphorylation events initiated by receptor, cytoplasmic and nuclear kinases and regulated by phosphatases are central to these processes. Mutation, hyper-activation, translocation and overexpression of protein tyrosine kinases are all associated with tumorigenesis. In addition to increasing proliferative rates and immortalizing cells, overexpression of tyrosine kinases can lead to morphological transformation and cause anchorage independence, contributing to the promotion of migratory ability and possibly the induction of metastases.

Certain compounds with structures based upon mimicry of ATP or phosphotyrosine have been shown to be effective kinase inhibitors. Those based upon phosphotyrosine have been demonstrated to be the more specific tyrosine kinase inhibitors. Because of their ability to inhibit tyrosine phosphorylation, these compounds may alter cell responses to growth factors or other process driven by tyrosine kinase activity, including unregulated growth as the result of tyrosine kinase overexpression, mutation, or translocation. Inhibition of tyrosine kinases occupying a central role in proliferative signaling pathways, or in pathways regulating cell cytoskeletal structure, even temporary or incomplete inhibition, may be sufficient to switch a cancerous cell from a proliferative cycle into programmed cell death, or apoptosis. Death by apoptosis is most often observed upon effective treatment with tyrosine kinase inhibitors.

Selective inhibition of specific tyrosine kinases offers a method of targeting cancerous cell growth with a high degree of specificity and minimal toxicity to normally growing cells and tissues. Thus, specific inhibitors of tyrosine kinases have great potential as clinical anti-cancer treatments. A number of small molecules which act as tyrosine kinase inhibitors have been identified. For example, certain phenyl acrylonitrile compounds have been described as tyrosine kinase inhibitors, effective to inhibit cell proliferation (see for example, U.S. Pat. No. 5,891,917, U.S. Pat. No. 5,217,999, U.S. Pat. No. 5,773,476, U.S. Pat. No. 5,935,993, U.S. Pat. No. 5,656,655, U.S. Pat. No. 5,677,329 and U.S. Pat. No. 5,789,427).

Inhibition of tyrosine kinases offers one mechanism by which cell proliferation can be inhibited. One of skill in the art will appreciate that other mechanisms of inhibition may also be involved.

There is a need in the art to identify compounds that inhibit cell proliferation, for treating cancer with increased site specificity, improved stability and solubility, and increased potency.

### SUMMARY OF THE INVENTION

A number of compounds have now been identified that inhibit abnormal cell proliferation, for example cancer cell growth, and/or inhibit angiogenesis.

According to another aspect of the present invention, there is provided a pharmaceutical composition comprising a compound of the invention and a pharmaceutically acceptable carrier or diluent, e.g., for treating a cancer selected from leukemia, non-small cell lung cancer, colon cancer, CNS cancer, melanoma, ovarian cancer, renal cancer, prostate cancer, and breast cancer.

In accordance with a further aspect of the present invention, there is provided a method for modulating cell proliferation, preferably inhibiting cell proliferation (e.g., a cancer selected from leukemia, non-small cell lung cancer, colon cancer, CNS cancer, melanoma, ovarian cancer, renal cancer, prostate cancer, and breast cancer), comprising administering an effective amount of a compound of the invention to a cell or animal in need thereof, e.g., diagnosed as having cancer and/or a tumor. The invention also includes use of a compound of the invention in the manufacture of a medicament to modulate cell proliferation, preferably inhibit cell proliferation (e.g., a cancer selected from leukemia, non-small cell lung cancer, colon cancer, CNS cancer, melanoma, ovarian cancer, renal cancer, prostate cancer, and breast cancer).

In a preferred embodiment the present invention provides a method of inhibiting the proliferation of a cancer cell comprising administering an effective amount of a compound of the invention to a cell or animal in need thereof. The cancer cell treated may be any type of cancer including leukemia, lymphoma, myeloma, metastatic carcinoma, sarcoma or any other malignant transformation or any other malignancy, including a cancer selected from leukemia, non-small cell lung cancer, colon cancer, CNS cancer, melanoma, ovarian cancer, renal cancer, prostate cancer, and breast cancer.

In another aspect, the invention provides a method of modulating tyrosine kinase activity in a cell by administering an effective amount of a compound of the invention. In a further aspect, the invention provides a method of inhibiting tyrosine kinase activity in a cell by administering an effective amount of a compound of the invention. The present invention also provides a use of a compound of the invention to modulate, preferably inhibit, tyrosine kinase activity. The present invention further provides a use of a compound of the invention to prepare a medicament to modulate tyrosine kinase activity, preferably inhibit tyrosine kinase activity. It is appreciated that the inhibition of cell growth by the compounds of the invention may be effected by other mechanisms.

In accordance with a further aspect of the present invention, there is provided a method for inhibiting angiogenesis, comprising administering an effective amount of a compound of the invention to a cell or animal in need thereof. The invention also includes a use of a compound of the invention in the manufacture of a medicament to inhibit angiogenesis.

Other features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### DETAILED DESCRIPTION OF THE INVENTION

A number of compounds have now been identified that are suitable for use to inhibit abnormal cell proliferation, for example cancer cell growth.

I. Compounds of the Invention

The present invention provides compounds of Formula I, and salts, solvates, or hydrates thereof: 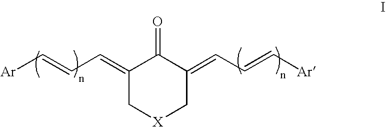
wherein
Ar and Ar′ are independently an optionally substituted phenyl ring;
each n is, independently for each occurrence, either 0 or 1;
X is selected from O, NR, and (CH₂)ₘ;
R is selected from H, OH, and alkyl, preferably C₁₋₄alkyl; and
m is an integer from 0 to 3, preferably 1 or 2.

In certain embodiments, substituents on Ar or Ar′ are selected from halo, cyano, sulfate, phosphate, SO₃H, P(═O)(OH)₂, NO₂, alkyl, haloalkyl (up to perhalo, e.g., perfluoro), OH, alkoxy, CH₃O(CH₂CH₂O)_{q}CO₂—, haloalkoxy (up to perhalo, e.g., perfluoro), —O—Si(alkyl)(alkyl)(alkyl), HO₂C—, alkylO₂C—, alkylC(O)—, alkylCO₂—, alkylOCO₂—, (R⁶)(R⁷)N—, R⁷C(O)N(R⁶)—, (R⁶)(R⁷)NCO—, (R⁶)(R⁷)NC(O)N(R⁶), (R⁶)(R⁷)NCO₂—, SH, alkylthio, and —O(CH₂)_{y}O—, wherein each y is, independently for each occurrence, an integer from 1 to 6, preferably 1 or 2, thereby forming a ring fused to the phenyl ring; R⁶ and R⁷ are each independently selected from hydrogen and C₁₋₆alkyl, or R⁶ and R⁷ together are —(CH₂)ₚY(CH₂)ₚ—, thereby forming a ring; each p is, independently for each occurrence, an integer from 1 to 3, preferably 1 or 2; and Y is selected from O, S, NH, NC₁₋₄alkyl, and CH₂.

In certain embodiments, Ar and Ar′ are independently substituted from one to three times at any of the 3-, 4-, or 5-positions (relative to the attachment to the rest of the molecule) with substituents selected from OH, alkyl, acyloxy, and alkoxy, preferably from OH, C₁₋₆alkyl, C₁₋₆alkylCO₂, and C₁₋₆alkoxy.

In certain alternative embodiments, Ar and Ar′ are independently substituted from one to three times at any of the 3, 4, or 5-positions (relative to the attachment to the rest of the molecule) with substituents selected from alkyl, acyloxy, and alkoxy, preferably from C₁₋₆alkyl, C₁₋₆alkylCO₂, and C₁₋₆alkoxy. In certain such embodiments, Ar and Ar′ are independently substituted from one to three times at any of the 3-, 4-, or 5-positions (relative to the attachment to the rest of the molecule) with substituents selected from C₁₋₆alkyl and C₁₋₆alkoxy, preferably C₁₋₆alkoxy, more preferably OCH₃. In certain embodiments, Ar and Ar′ are each independently substituted at the 3, 4, and 5-positions. In certain such embodiments, the molecule is symmetrical, e.g., both values of n are equal, and Ar and Ar′ are identical.

In certain preferred embodiments, n is 1 for each occurrence and X is NR, preferably N(lower alkyl), most preferably N(C₂H₅). In certain such embodiments, at least one of Ar′ and Ar is unsubstituted, monosubstituted, tetrasubstituted, or pentasubstituted, and/or at least one of Ar′ and Ar includes a substituent other than OH or OMe, such as a substituent selected from halo, cyano, sulfate, phosphate, SO₃H, P(═O)(OH)₂, NO₂, alkyl, haloalkyl (up to perhalo, e.g., perfluoro), C₂₋₁₀alkoxy (preferably C₃₋₁₀alkoxy), CH₃O(CH₂CH₂O)_{q}CO₂—, haloalkoxy (up to perhalo, e.g., perfluoro), O—Si(alkyl)(alkyl)(alkyl), HO₂C—, alkylO₂C—, alkylC(O)—, alkylCO₂, alkylOCO₂, (R⁶)(R⁷)N—, R⁷C(O)N(R⁶)—, (R⁶)(R⁷)NC(O)—, (R⁶)(R⁷)NC(O)N(R⁶), (R⁶)(R⁷)NCO₂, SH, alkylthio, or —O(CH₂)_{y}O—.

In certain embodiments, each occurrence of n is 1. In certain alternative embodiments, each occurrence of n is 0.

In certain embodiments where Ar and Ar′ are both substituted at each of the 3-, 4-, and 5-positions, relative to the point of attachment to the rest of the molecule, and the 3- and 4-positions are each substituted with OCH₃, then the substituent at the 4-position is not OH; and

In certain embodiments where Ar and Ar′ are both substituted with OCH₃ at the 4-position relative to the point of attachment to the rest of the molecule, then at least one of Ar and Ar′ must be substituted at least one of the 3- and 5-positions.

Exemplary compounds of Formula I are the following (See Youssef, et al., Arch. Pharm. Med. Chem. 2004, 337, 42-54 and Youssef, et al., Arch. Pharm. Med. Chem. 2005, 338, 181-189, the disclosures of which are incorporated herein in their entirety):

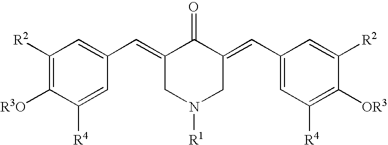

| R¹ | R² | R³ | R⁴ |
|---|---|---|---|
| CH₃ | H | H | H |
| CH₃ | H | CH₃ | H |
| CH₃ | H | H | OCH₃ |
| CH₃ | H | H | OC₂H₅ |
| CH₃ | CH₃ | H | CH₃ |
| CH₃ | OCH₃ | H | OCH₃ |
| C₂H₅ | H | H | H |
| C₂H₅ | H | CH₃ | H |
| C₂H₅ | H | H | OCH₃ |
| C₂H₅ | H | H | OC₂H₅ |
| C₂H₅ | CH₃ | H | CH₃ |
| C₂H₅ | OCH₃ | H | OCH₃ |
| C₃H₇ | H | H | H |
| C₃H₇ | H | CH₃ | H |
| C₃H₇ | H | H | OCH₃ |
| C₃H₇ | H | H | OC₂H₅ |
| CH₃ | H | H | H |
| CH₃ | H | CH₃ | H |
| CH₃ | H | H | OCH₃ |
| CH₃ | H | H | OC₂H₅ |
| C₂H₅ | H | H | H |
| C₂H₅ | H | CH₃ | H |
| C₂H₅ | H | H | OCH₃ |
| C₃H₇ | H | H | H |
| C₃H₇ | H | H | OCH₃ |

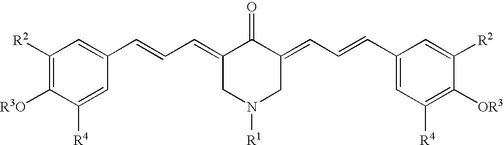

| R¹ | R² | R³ | R⁴ |
|---|---|---|---|
| CH₃ | CH₃ | H | OCH₃ |
| C₂H₅ | CH₃ | H | OCH₃ |

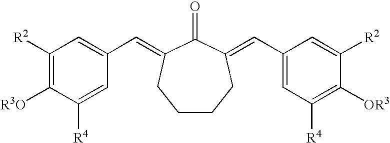

| R² | R³ | R⁴ |
|---|---|---|
| H | H | H |
| H | CH₃ | H |
| H | H | OCH₃ |
| H | H | OC₂H₅ |
| CH₃ | H | CH₃ |
| H | H | H |
| H | H | OH |
| H | H | OCH₃ |

The present invention provides pharmaceutical compositions, comprising a compound of Formula I or a salt, solvate, or hydrate thereof and a pharmaceutically acceptable carrier: 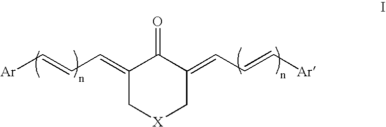
wherein
Ar and Ar′ are independently a phenyl ring optionally substituted with one or more substituents selected from halo, cyano, sulfate, phosphate, SO₃H, P(═O)(OH)₂, NO₂, alkyl, haloalkyl (up to perhalo, e.g., perfluoro), OH, alkoxy, CH₃O(CH₂CH₂O)_{q}CO₂—, haloalkoxy (up to perhalo, e.g., perfluoro), —O—Si(alkyl)(alkyl)(alkyl), HO₂C—, alkylO₂C—, alkylC(O)—, alkylCO₂—, alkylOCO₂—, (R⁶)(R⁷)N—, R⁷C(O)N(R⁶)—, (R⁶)(R⁷)NCO—, (R⁶)(R⁷)NC(O)N(R⁶), (R⁶)(R⁷)NCO₂—, SH, alkylthio, and —O(CH₂)_{y}O—;
each y is, independently for each occurrence, an integer from 1 to 6, preferably 1 or 2, thereby forming a ring fused to the phenyl ring;
R⁶ and R⁷ are each independently selected from hydrogen and C₁₋₆alkyl, or R⁶ and R⁷ together are —(CH₂)ₚY(CH₂)ₚ—, thereby forming a ring;
each p is, independently for each occurrence, an integer from 1 to 3, preferably 1 or 2;
Y is selected from O, S, NH, NC₁₋₆alkyl, and CH₂;
each n is, independently for each occurrence, either 0 or 1;
X is selected from O, NR, and (CH₂)ₘ;
R is selected from H, OH, and alkyl, preferably C₁₋₆alkyl; and
m is an integer from 0 to 3, preferably 1 or 2;
provided that when Ar and Ar′ are both substituted at each of the 3-, 4-, and 5-positions, relative to the point of attachment to the rest of the molecule, and the 3- and 4-positions are each substituted with OCH₃, then the substituent at the 4-position is not OH.

In certain embodiments, when Ar and Ar′ are both substituted with OCH₃ at the 4-position relative to the point of attachment to the rest of the molecule, then at least one of Ar and Ar′ must be further substituted, e.g., at at least one of the 3- and 5-positions.

In certain embodiments, Ar and Ar′ are independently substituted from one to three times at any of the 3-, 4-, or 5-positions (relative to the attachment to the rest of the molecule) with substituents selected from OH, alkyl, acyloxy, and alkoxy, preferably from OH, C₁₋₆alkyl, C₁₋₆alkylCO₂, and C₁₋₆alkoxy.

In certain alternative embodiments, Ar and Ar′ are independently substituted from one to three times at any of the 3, 4, or 5-positions (relative to the attachment to the rest of the molecule) with substituents selected from alkyl, acyloxy, and alkoxy, preferably from C₁₋₆alkyl, C₁₋₆alkylCO₂, and C₁₋₆alkoxy. In certain such embodiments, Ar and Ar′ are independently substituted from one to three times at any of the 3-, 4-, or 5-positions (relative to the attachment to the rest of the molecule) with substituents selected from C₁₋₆alkyl and C₁₋₆alkoxy, preferably C₁₋₆alkoxy, more preferably OCH₃. In certain embodiments, Ar and Ar′ are each independently substituted at the 3, 4, and 5-positions. In certain such embodiments, the molecule is symmetrical, e.g., both values of n are equal, and Ar and Ar′ are identical.

In certain preferred embodiments, n is 1 for each occurrence and X is NR, preferably N(lower alkyl), most preferably N(C₂H₅). In certain such embodiments, at least one of Ar′ and Ar is unsubstituted, monosubstituted, tetrasubstituted, or pentasubstituted, and/or at least one of Ar′ and Ar includes a substituent other than OH or OMe, such as a substituent selected from halo, cyano, sulfate, phosphate, SO₃H, P(═O)(OH)₂, NO₂, alkyl, haloalkyl (up to perhalo, e.g., perfluoro), C₂₋₁₀alkoxy (preferably C₃₋₁₀alkoxy), CH₃O(CH₂CH₂O)_{q}CO₂—, haloalkoxy (up to perhalo, e.g., perfluoro), O—Si(alkyl)(alkyl)(alkyl), HO₂C—, alkylO₂C—, alkylC(O)—, alkylCO₂, alkylOCO₂, (R⁶)(R⁷)N—, R⁷C(O)N(R⁶)—, (R⁶)(R⁷)NC(O)—, (R⁶)(R⁷)NC(O)N(R⁶), (R⁶)(R⁷)NCO₂, SH, alkylthio, or —O(CH₂)_{y}O—.

In certain embodiments, each occurrence of n is 1. In certain alternative embodiments, each occurrence of n is 0 .

The present invention also provides pharmaceutical compositions, comprising a compound of Formula Ia or a salt, solvate, or hydrate thereof and a pharmaceutically acceptable carrier: 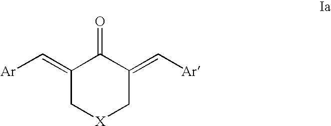
wherein
Ar and Ar′ are independently a phenyl ring substituted one to three times with substituents selected from alkyl, haloalkyl (up to perhalo, e.g., perfluoro), alkoxy, and haloalkoxy (up to perhalo, e.g., perfluoro), preferably alkyl or alkoxy, more preferably alkoxy;
X is NR; and
R is selected from H and alkyl, preferably C₁₋₆alkyl, more preferably methyl or ethyl.

In certain embodiments, when Ar and Ar′ are both substituted with OCH₃ at the 4-position relative to the point of attachment to the rest of the molecule, then at least one of Ar and Ar′ must be further substituted, e.g., at at least one of the 3- and 5-positions.

In certain embodiments, the substituents on Ar and Ar′, if any, are located at the 3-, 4-, and/or 5-positions relative to the point of attachment to the rest of the molecule. In certain embodiments, one or both of Ar and Ar′ bears a substituent at the 3-position relative to the point of attachment to the rest of the molecule.

In certain embodiments, Ar and Ar′ are substituted at the 3-, 4-, and 5-positions relative to the point of attachment to the rest of the molecule, wherein the substituents are independently selected from alkyl and alkoxy. In certain such embodiments, Ar and Ar′ are substituted at the 3-, 4-, and 5-positions, wherein the substituents are independently alkoxy, preferably all of the substituents are methoxy.

In certain embodiments, the invention relates to a pharmaceutical composition comprising a compound of Formula I or a salt, solvate, or hydrate thereof and a pharmaceutically acceptable carrier, wherein the compound of Formula I is Compound A:

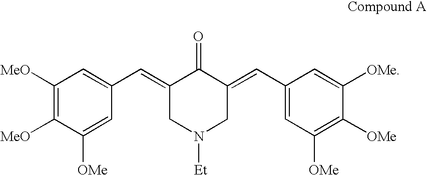

The present invention also provides a method for the treatment of cancer, comprising administering a compound of Formula I, or a salt, solvate, or hydrate thereof: 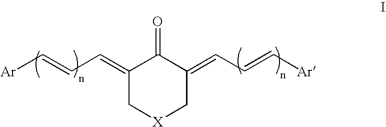
wherein
Ar and Ar′ are independently a phenyl ring optionally substituted with one or more substituents selected from halo, cyano, sulfate, phosphate, SO₃H, P(═O)(OH)₂, NO₂, alkyl, haloalkyl (up to perhalo, e.g., perfluoro), OH, alkoxy, CH₃O(CH₂CH₂O)_{q}CO₂—, haloalkoxy (up to perhalo, e.g., perfluoro), —O—Si(alkyl)(alkyl)(alkyl), HO₂C—, alkylO₂C—, alkylC(O)—, alkylCO₂—, alkylOCO₂—, (R⁶)(R⁷)N—, R⁷C(O)N(R⁶)—, (R⁶)(R⁷)NCO—, (R⁶)(R⁷)NC(O)N(R⁶), (R⁶)(R⁷)NCO₂—, SH, alkylthio, and —O(CH₂)_{y}O—;
each y is, independently for each occurrence, an integer from 1 to 6, preferably 1 or 2, thereby forming a ring fused to the phenyl ring;
R⁶ and R⁷ are each independently selected from hydrogen and C₁₋₆alkyl, or R⁶ and R⁷ together are —(CH₂)ₚY(CH₂)ₚ—, thereby forming a ring;
each p is, independently for each occurrence, an integer from 1 to 3, preferably 1 or 2;
Y is selected from O, S, NH, NC₁₋₆alkyl, and CH₂;
each n is, independently for each occurrence, either 0 or 1;
X is selected from O, NR, and (CH₂)ₘ;
R is selected from H, OH, and alkyl, preferably C₁₋₆alkyl; and
m is an integer from 0 to 3, preferably 1 or 2.

In certain embodiments, when Ar and Ar′ are both substituted with OCH₃ at the 4-position relative to the point of attachment to the rest of the molecule, then at least one of Ar and Ar′ must be further substituted, e.g., at at least one of the 3- and 5-positions.

In certain embodiments, Ar and Ar′ are independently substituted from one to three times at any of the 3, 4, or 5-positions (relative to the attachment to the rest of the molecule) with substituents selected from OH, alkyl, acyloxy, and alkoxy, preferably from OH, C₁₋₆alkyl, C₁₋₆alkylCO₂—, and C₁₋₆alkoxy.

In certain alternative embodiments, Ar and Ar′ are independently substituted from one to three times at any of the 3, 4, or 5-positions (relative to the attachment to the rest of the molecule) with substituents selected from alkyl, acyloxy, and alkoxy, preferably from C₁₋₆alkyl, C₁₋₆alkylCO₂—, and C₁₋₆alkoxy. In certain such embodiments, Ar and Ar′ are independently substituted from one to three times at any of the 3, 4, or 5-positions (relative to the attachment to the rest of the molecule) with substituents selected from C₁₋₆alkyl and C₁₋₆alkoxy, preferably C₁₋₆alkoxy, more preferably OCH₃. In certain embodiments, Ar and Ar′ are each independently substituted at the 3, 4, and 5-positions. In certain such embodiments, the molecule is symmetrical, e.g., both values of n are equal, and Ar and Ar′ are identical.

In certain preferred embodiments, n is 1 for each occurrence and X is NR, preferably N(lower alkyl), most preferably N(C₂H₅). In certain such embodiments, at least one of Ar′ and Ar is unsubstituted, monosubstituted, tetrasubstituted, or pentasubstituted, and/or at least one of Ar′ and Ar includes a substituent other than OH or OMe, such as a substituent selected from halo, cyano, sulfate, phosphate, SO₃H, P(═O)(OH)₂, NO₂, alkyl, haloalkyl (up to perhalo, e.g., perfluoro), C₂₋₁₀alkoxy (preferably C₃₋₁₀alkoxy), CH₃O(CH₂CH₂O)_{q}CO₂—, haloalkoxy (up to perhalo, e.g., perfluoro), O—Si(alkyl)(alkyl)(alkyl), HO₂C—, alkylO₂C—, alkylC(O)—, alkylCO₂, alkylOCO₂, (R⁶)(R⁷)N—, R⁷C(O)N(R⁶)—, (R⁶)(R⁷)NC(O)—, (R⁶)(R⁷)NC(O)N(R⁶), (R⁶)(R⁷)NCO₂, SH, alkylthio, or —O(CH₂)_{y}O—.

In certain embodiments, n is 1 for each occurrence. In other embodiments, n is 0 for each occurrence.

The present invention also provides a method for the treatment of cancer, comprising administering a compound of Formula Ia or a salt, solvate, or hydrate thereof: 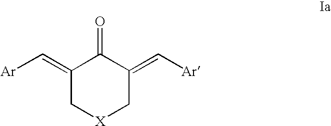
wherein
Ar and Ar′ are independently a phenyl ring substituted one to three times with substituents selected from alkyl, haloalkyl (up to perhalo, e.g., perfluoro), alkoxy, and haloalkoxy (up to perhalo, e.g., perfluoro), preferably alkyl or alkoxy, more preferably alkoxy;
X is NR; and
R is selected from H and alkyl, preferably C₁₋₆alkyl, more preferably methyl or ethyl.

In certain embodiments, when Ar and Ar′ are both substituted with OCH₃ at the 4-position relative to the point of attachment to the rest of the molecule, then at least one of Ar and Ar′ must be further substituted, e.g., at at least one of the 3- and 5-positions.

In certain embodiments, the substituents on Ar and Ar′, if any, are located at the 3-, 4-, and/or 5-positions relative to the point of attachment to the rest of the molecule. In certain embodiments, one or both of Ar and Ar′ bears a substituent at the 3-position relative to the point of attachment to the rest of the molecule.

In certain embodiments, Ar and Ar′ are substituted at the 3-, 4-, and 5-positions relative to the point of attachment to the rest of the molecule, wherein the substituents are independently selected from alkyl and alkoxy. In certain such embodiments, Ar and Ar′ are substituted at the 3-, 4-, and 5-positions, wherein the substituents are independently alkoxy, preferably all of the substituents are methoxy.

In certain embodiments, the invention relates to a method for the treatment of cancer, comprising administering a compound of Formula I, or a salt, solvate, or hydrate thereof, wherein the compound of Formula I is Compound A:

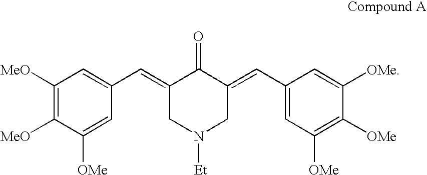

The present invention also provides compounds of Formula II, and salts, solvates, or hydrates thereof: 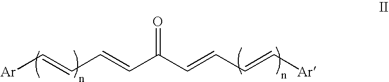
wherein
Ar and Ar′ each independently represents a phenyl ring optionally substituted with one or more substituents selected from halo, cyano, sulfate, phosphate, SO₃H, P(═O)(OH)₂, NO₂, alkyl, haloalkyl (up to perhalo, e.g., perfluoro), OH, alkoxy, CH₃O(CH₂CH₂O)_{q}CO₂—, haloalkoxy (up to perhalo, e.g., perfluoro), —O—Si(alkyl)(alkyl)(alkyl), HO₂C—, alkylO₂C—, alkylCO—, alkylCO₂—, alkylOCO₂—, (R⁶)(R⁷)N—, R⁷CON(R⁶), (R⁶)(R⁷)NC(O)—, (R⁶)(R⁷)NC(O)N(R⁶), R⁶R⁷NCO₂—, SH, alkylthio, or —(CH₂)_{y}O—;
each y is, independently for each occurrence, an integer from 1 to 6, preferably 1 or 2, thereby forming a ring fused to the phenyl ring;
R⁶ and R⁷ are each independently selected from hydrogen and C₁₋₆alkyl, or R⁶ and R⁷ together are —(CH₂)ₚY(CH₂)ₚ—, thereby forming a ring;
each p is, independently for each occurrence, an integer from 1 to 3, preferably 1 or 2,
Y is selected from O, S, NH, N—C₁₋₆alkyl, and CH₂;
n independently for each occurrence represents either 0 or 1.

In certain embodiments, Ar and/or Ar′ is substituted at either a meta position, a para position, or both (relative to the attachment to the rest of the molecule) by substituents selected from OH, alkyl, acyloxy, and alkoxy, preferably from OH, C₁₋₆alkyl, C₁₋₆alkylCO₂, and C₁₋₆alkoxy. In certain embodiments, the substituents are selected from halo, cyano, sulfate, phosphate, SO₃H, P(═O)(OH)₂, NO₂, C₁₋₆alkyl, C₁₋₄haloalkyl (up to perhalo, e.g., perfluoro), OH, C₁₋₆alkoxy, CH₃O(CH₂CH₂O)_{q}CO₂, C₁₋₆haloalkoxy (up to perhalo, e.g., perfluoro), O—Si(C₁₋₆alkyl)(C₁₋₆alkyl)(C₁₋₆alkyl), HO₂C—, C₁₋₆alkylO₂C—, C₁₋₆alkylC(O)—, C₁₋₆alkylCO₂, C₁₋₆alkylOCO₂—, (R⁵O)₂PO₂, (R⁶)(R⁷)N—, R⁷C(O)N(R⁶)—, (R⁶)(R⁷)NC(O)—, (R⁶)(R⁷)NC(O)N(R⁶)—, (R⁶)(R⁷)NCO₂—, SH, C₁₋₆alkylthio, or —O(CH₂)_{y}O—. In certain embodiments, at least one of Ar and Ar′, preferably both, is substituted with three different substituents, such as OH, C₁₋₆alkyl, and C₁₋₆alkoxy. In certain embodiments, the molecule is symmetrical, e.g., both values of n are equal, and Ar and Ar′ are identical. In certain embodiments, both occurrences of n are 1.

In certain embodiments, the invention relates to a pharmaceutical composition, comprising a compound of formula II, or a salt, solvate, or hydrate thereof, and a pharmaceutically acceptable carrier.

In certain embodiments, the invention relates to a method of treating cancer, comprising administering a compound Formula II or a salt, solvate, or hydrate thereof.

Exemplary compounds of Formula II are the following (See Youssef, et al., Arch. Pharm. Med. Chem. 2004, 337, 42-54 and Youssef, et al., Arch. Pharm. Med. Chem. 2005, 338, 181-189, the disclosures of which are incorporated herein in their entirety):

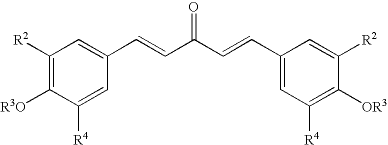

| R² | R³ | R⁴ |
|---|---|---|
| H | H | H |
| H | CH₃ | H |
| H | H | OCH₃ |
| H | H | OC₂H₅ |
| CH₃ | H | CH₃ |
| OCH₃ | H | OCH₃ |
| H | H | H |
| H | H | OCH₃ |

The present invention contemplates that all combinations of the particular embodiments and preferences disclosed above are within the scope of the invention.

Some compounds of the invention may have at least one asymmetric center. Where the compounds according to the invention have one asymmetric center, the may exist as enantiomers. Where the compounds of the invention possess two or more asymmetric centers, they may additionally exist as diastereomers. It is to be understood that all such isomers and mixtures thereof in any proportion are encompassed within the scope of the present invention.

In certain embodiments of each of the above formulae, Ar, Ar′, or both are substituted at a para position with an oxygen-based substituent such as hydroxy or alkoxy, such as a hydroxyl group or an ester or other prodrug form thereof. In certain such embodiments, Ar, Ar′, or both further include additional oxygen substituents, such as hydroxy, alkoxy, and protected forms thereof.

In certain embodiments, the compounds of Example I below are expressly excluded from the above generic formulae.

The present invention includes radiolabeled forms of compounds of the invention, for example, compounds of the invention labeled by incorporation within the structure ³H or ¹⁴C or a radioactive halogen such as ¹²⁵I.

In certain embodiments, compounds of the invention may be useful in the inhibition of the growth of cancer cells, in particular hematopoietic cell malignancies, acute lymphoblastic leukemia, Philadelphia positive leukemia and acute myeloid leukemia. In certain embodiments, the cancer is selected from leukemia, non-small cell lung cancer, colon cancer, CNS cancer, melanoma, ovarian cancer, renal cancer, prostate cancer, and breast cancer.

In another embodiment, compounds of the invention may be useful as a method for the treatment of cancer, in particular hematopoietic cell malignancies, acute lymphoblastic leukemia, Philadelphia positive leukemia and acute myeloid leukemia.

In other embodiments, compounds of the invention may be useful in treating other conditions involving aberrant or abnormal cell proliferation. Other cell proliferative disorders that may be treated by the present invention include inflammatory diseases, allergies, autoimmune disease, graft rejection psoriasis, restenosis, atherosclerosis, and any other disorder wherein it is desirable to inhibit, prevent or suppress cell growth. Compounds of the invention may be tested for their efficacy in a particular cell proliferation disorder using assays and techniques known to those of skill in the art. For example, the following references provide assays for various conditions including rheumatoid arthritis, allergies, and psoriasis: C. S. Kasyapa et al., J. Immun., 1999, 163, 8236; T. Adachi et al., J. Immun., 1999, 163, 939; R. Üchert, J. Immun., 2000, 165, 224; A. H. Enk, International Archives of Allergy and Immunology, 2000, 123, 275.

In an additional embodiment, compounds of the invention may be useful in modulating tyrosine kinase activity, including the inhibition of tyrosine kinase activity for the treatment of various conditions such as proliferative disorders as mentioned above. Accordingly, the invention may be useful as a method of modulating tyrosine kinase activity by administering a therapeutically effective amount of a compound of the invention to a cell or animal in need thereof. In a further aspect, the invention may be useful as a method of inhibiting tyrosine kinase activity by administering an effective amount of a compound of the invention to a cell or animal in need thereof.

While the compounds of the invention may act by inhibiting tyrosine kinase activity, one of skill in the art will appreciate that other modes or mechanisms of action for the compounds of the invention are possible.

Thus, compounds of the invention may be useful for the promotion of myelopoiesis. In other embodiments the invention may be useful as a method of promoting myelopoiesis in vivo, ex vivo, or in vitro. In one aspect, the invention relates to a method of promoting myelopoiesis comprising administering an effective amount of one or more of these myelopoiesis-promoting compounds to hematopoietic cell or an animal in need thereof, wherein the term “cell” includes a plurality of cells, and administration to a cell includes in vivo, ex vivo, and in vitro treatment.

In some embodiments, the hematopoietic cell is hematopoietic stem cell and the animal is a human patient. In specific embodiments, the compounds are administered to a human patient suffering from, or at risk of primary or secondary neutropenia, including chemotherapy or drug induced neutropenia, neutropenia secondary to malignancy, including G-CSF responsive malignancies. In other embodiments, the compound(s) is administered to a human patient at risk of or suffering from aplastic anemia or aplasia. In other embodiments, the animal is a human donor of bone marrow cells or peripheral blood stem cells. In other embodiments, one or more of these myelopoiesis-promoting compounds is administered to a human patient in need of bone marrow cell or peripheral blood stem cell transplant before or after the transplant.

In another aspect, the invention provides a method of promoting myelopoiesis ex vivo comprising administering an effective amount of one or more of these myelopoiesis-promoting compounds to a hematopoietic cell. In one embodiment, the cell is hematopoietic stem cell. In specific embodiments, the hematopoietic cell is from the bone marrow or peripheral blood stem cells of a donor, or the bone marrow or peripheral blood stem cells of a patient in need of autologous bone marrow or peripheral blood stem cell transplant.

In another aspect, the invention provides a method of treating a patient suffering from or at risk of neutropenia, aplastic anemia or aplasia, comprising administering an effective amount of one or more of these myelopoiesis-promoting compounds to the patient. In another aspect, the invention provides a method of treating a patient suffering from or at risk of neutropenia, aplastic anemia or aplasia, comprising introducing hematopoietic cells to the patient wherein one or more of these myelopoiesis-promoting compounds has been administered to the cells ex vivo in an amount effective to promote myelopoiesis. The hematopoietic cells may be from the bone marrow or peripheral blood stem cells of a donor or of the patient.

In another aspect, the invention relates to use of one or more of these myelopoiesis-promoting compounds to promote myelopoiesis. The invention also relates to use of one or more of these myelopoiesis-promoting compounds for preparing a medicament to promote myelopoiesis. Yet in another aspect, the invention relates to use of one or more of these myelopoiesis-promoting compounds to treat neutropenia, aplastic anemia or aplasia, and the use of one or more of these myelopoiesis-promoting compounds to prepare a medicament to treat neutropenia aplastic anemia or aplasia.

In another aspect, the invention provides a kit comprising one or more of these myelopoiesis-promoting compounds and instructions for use, including to promote myelopoiesis and to treat neutropenia, aplastic anemia, or aplasia.

The present invention also includes a use of a compound or composition of the invention in order to inhibit cell proliferation, preferably cancer cell proliferation. The present invention further includes a use of a compound or a composition of the invention to prepare a medicament to inhibit cell proliferation, preferably cancer cell proliferation.

In certain embodiments, a compound of the invention is administered to a patient diagnosed as having cancer, such as a tumor. In certain such embodiments, the method further comprises monitoring the patient to detect changes in the size or aggressiveness of the cancer or tumor. In certain embodiments, the method is performed on a patient prior to surgical removal of a tumor; in other embodiments, the method is performed after the tumor has been surgically removed.

The compounds of the invention can be used alone or in combination with other agents that modulate tyrosine kinase activity or in combination with other types of treatment (which may or may not modulate tyrosine kinase activity) for cell proliferative disorders. Agents known in the art that inhibit tyrosine kinase activity include, but are not limited to, antisense nucleic acid and ribozymes targeted to nucleic acid encoding a receptor tyrosine kinase, antibodies able to modulate tyrosine kinase activity and other small molecule tyrosine kinase inhibitors such as those described in U.S. Pat. No. 5,891,917, U.S. Pat. No. 5,217,999, U.S. Pat. No. 5,773,476, U.S. Pat. No. 5,935,993, U.S. Pat. No. 5,656,655, U.S. Pat. No. 5,677,329 and U.S. Pat. No. 5,789,427. There are various examples of other types of treatment for cell proliferative disorders currently used to treat different types of cancers. The general treatments are based on the cancer type and do not specifically target tyrosine kinase activity. In a particular aspect of the present invention, the compounds of the invention may be used in combination with other therapies and therapeutics to treat leukemia.

Similarly, compounds of the invention can be used in conjunction with other cancer therapies, including radiation, chemotherapy (including antineoplastics, antiangiogenics, and other compounds that directly or indirectly affect tumor growth and/or survival), thermal ablation, radiofrequency ablation, cryosurgery, ultrasound, microwave irradiation, or other suitable treatments.

II. Definitions

The term “alkyl” refers to saturated aliphatic groups, including straight-chain alkyl groups, branched-chain alkyl groups, cycloalkyl (alicyclic) groups, alkyl-substituted cycloalkyl groups, and cycloalkyl-substituted alkyl groups. In preferred embodiments, a straight chain or branched chain alkyl has 30 or fewer carbon atoms in its backbone (e.g., C₁₋₃₀ for straight chains, C₃₋₃₀ for branched chains), 20 or fewer, 10 or fewer, or even 6 or fewer.

Moreover, the term “alkyl” as used throughout the specification, examples, and claims is intended to include both unsubstituted and substituted alkyl groups, the latter of which refers to alkyl moieties having substituents replacing a hydrogen on one or more carbons of the hydrocarbon backbone, including haloalkyl groups such as trifluoromethyl and 2,2,2-trifluoroethyl, etc.

The term “C_{x-y}” when used in conjunction with a chemical moiety, such as, acyl, acyloxy, alkyl, alkenyl, alkynyl, or alkoxy is meant to include groups that contain from x to y carbons in the chain. C₀alkyl indicates a hydrogen where the group is in a terminal position, a bond if internal. A C₁₋₆alkyl group, for example, contains from one to six carbon atoms in the chain.

The terms “halo” and “halogen” as used herein means halogen and includes chloro, fluoro, bromo, and iodo.

The term “haloalkyl”, as used herein, refers to an alkyl group substituted with a halogen.

To “inhibit” or “suppress” or “reduce” a function or activity, such as cancer cell proliferation, is to reduce the function or activity when compared to otherwise same conditions except for a condition or parameter of interest, or alternatively, as compared to another conditions.

The term “lower” when used in conjunction with a chemical moiety, such as, acyl, acyloxy, alkyl, alkenyl, alkynyl, or alkoxy is meant to include groups where there are ten or fewer atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The term “modulate” as used herein includes the inhibition or suppression of a function or activity (such as cell proliferation) as well as the enhancement of a function or activity.

“Palliating” a disease or disorder means that the extent and/or undesirable clinical manifestations of a disorder or a disease state are lessened and/or time course of the progression is slowed or lengthened, as compared to not treating the disorder.

The phrase “pharmaceutically acceptable” is employed herein to refer to those ligands, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material, involved in carrying or transporting the subject chemical from one organ or portion of the body, to another organ or portion of the body. Each carrier must be “acceptable” in the sense of being compatible with the other ingredients of the formulation and not injurious to the patient. Some examples of materials which can serve as pharmaceutically acceptable carriers include: (1) sugars, such as lactose, glucose, and sucrose; (2) starches, such as corn starch and potato starch; (3) cellulose, and its derivatives, such as sodium carboxymethyl cellulose, ethyl cellulose, and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil, and soybean oil; (10) glycols, such as propylene glycol; (11) polyols, such as glycerin, sorbitol, mannitol, and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations. In certain embodiments, pharmaceutical compositions of the present invention are non-pyrogenic, i.e., do not induce significant temperature elevations when administered to a patient.

The term “pharmaceutically acceptable salt” refers to the relatively non-toxic, inorganic and organic acid addition salts of the inhibitor(s). These salts can be prepared in situ during the final isolation and purification of the inhibitor(s), or by separately reacting a purified inhibitor(s) in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, naphthylate, mesylate, glucoheptonate, lactobionate, amino acids, and laurylsulphonate salts, and the like. (See, for example, Berge et al. (1977) “Pharmaceutical Salts”, J. Pharm. Sci. 66:1-19)

In other cases, the inhibitors useful in the methods of the present invention may contain one or more acidic functional groups and, thus, are capable of forming pharmaceutically acceptable salts with pharmaceutically acceptable bases. The term “pharmaceutically acceptable salts” in these instances refers to the relatively non-toxic inorganic and organic base addition salts of an inhibitor(s). These salts can likewise be prepared in situ during the final isolation and purification of the inhibitor(s), or by separately reacting the purified inhibitor(s) in its free acid form with a suitable base, such as the hydroxide, carbonate, or bicarbonate of a pharmaceutically acceptable metal cation, with ammonia, or with a pharmaceutically acceptable organic primary, secondary, or tertiary amine. Representative alkali or alkaline earth salts include the lithium, sodium, potassium, calcium, magnesium, and aluminum salts, and the like. Representative organic amines useful for the formation of base addition salts include ethylamine, diethylamine, ethylenediamine, ethanolamine, diethanolamine, piperazine, and the like (see, for example, Berge et al., supra).

The term “prodrug” encompasses compounds that, under physiological conditions, are converted into therapeutically active agents. A common method for making a prodrug is to include selected moieties that are hydrolyzed under physiological conditions to reveal the desired molecule. In other embodiments, the prodrug is converted by an enzymatic activity of the host animal.

The term “solvate” as used herein means a compound or a pharmaceutically acceptable salt of a compound wherein molecules of a suitable solvent are incorporated in the crystal lattice. A suitable solvent is physiologically tolerable at the dosage administered. Examples of suitable solvents are ethanol, water and the like. When water is the solvent, the molecule is referred to as a “hydrate”.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

A “therapeutically effective amount” of a compound with respect to the subject method of treatment, refers to an amount of the compound(s) in a preparation which, when administered as part of a desired dosage regimen (to a mammal, preferably a human) alleviates a symptom, ameliorates a condition, or slows the onset of disease conditions according to clinically acceptable standards for the disorder or condition to be treated or the cosmetic purpose, e.g., at a reasonable benefit/risk ratio applicable to any medical treatment.

As used herein, the term “treating” or “treatment” includes reversing, reducing, or arresting the symptoms, clinical signs, and underlying pathology of a condition in manner to improve or stabilize a subject's condition. As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e., not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

III. Administration

The compounds of the invention are preferably formulated into pharmaceutical compositions for administration to human subjects in a biologically compatible form suitable for administration in vivo. Accordingly, in another aspect, the present invention provides a pharmaceutical composition comprising a compound of the invention in admixture with a suitable diluent or carrier.

The compositions containing the compounds of the invention can be prepared by known methods for the preparation of pharmaceutically acceptable compositions which can be administered to subjects, such that an effective quantity of the active substance is combined in a mixture with a pharmaceutically acceptable vehicle. Suitable vehicles are described, for example, in Remington's Pharmaceutical Sciences (Remington's Pharmaceutical Sciences, Mack Publishing Company, Easton, Pa., USA 1985).

Wetting agents, emulsifiers, and lubricants, such as sodium lauryl sulfate and magnesium stearate, as well as coloring agents, release agents, coating agents, sweetening, flavoring, and perfuming agents, preservatives and antioxidants can also be present in the compositions.

Examples of pharmaceutically acceptable antioxidants include: (1) water soluble antioxidants, such as ascorbic acid, cysteine hydrochloride, sodium bisulfate, sodium metabisulfite, sodium sulfite, and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouthwashes, and the like, each containing a predetermined amount of an inhibitor(s) as an active ingredient. A compound may also be administered as a bolus, electuary or paste.

In solid dosage forms for oral administration (capsules, tablets, pills, dragees, powders, granules, and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose, and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets, and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols, and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered peptide or peptidomimetic moistened with an inert liquid diluent.

Tablets, and other solid dosage forms, such as dragees, capsules, pills, and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes, and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration include pharmaceutically acceptable emulsions, microemulsions, solutions, suspensions, syrups, and elixirs. In addition to the active ingredient, the liquid dosage forms may contain inert diluents commonly used in the art, such as, for example, water or other solvents, solubilizing agents, and emulsifiers such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols, and fatty acid esters of sorbitan, and mixtures thereof.

Besides inert diluents, the oral compositions can also include adjuvants such as wetting agents, emulsifying and suspending agents, sweetening, flavoring, coloring, perfuming, and preservative agents.

Suspensions, in addition to the active inhibitor(s) may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing one or more inhibitor(s) with one or more suitable nonirritating excipients or carriers comprising, for example, cocoa butter, polyethylene glycol, a suppository wax or a salicylate, which is solid at room temperature, but liquid at body temperature and, therefore, will melt in the rectum or vaginal cavity and release the active agent.

Formulations which are suitable for vaginal administration also include pessaries, tampons, creams, gels, pastes, foams, or spray formulations containing such carriers as are known in the art to be appropriate.

Dosage forms for the topical or transdermal administration of an inhibitor(s) include powders, sprays, ointments, pastes, creams, lotions, gels, solutions, patches, and inhalants. The active component may be mixed under sterile conditions with a pharmaceutically acceptable carrier, and with any preservatives, buffers, or propellants which may be required.

The ointments, pastes, creams, and gels may contain, in addition to inhibitor(s), excipients, such as animal and vegetable fats, oils, waxes, paraffins, starch, tragacanth, cellulose derivatives, polyethylene glycols, silicones, bentonites, silicic acid, talc, and zinc oxide, or mixtures thereof.

Powders and sprays can contain, in addition to an inhibitor(s), excipients such as lactose, talc, silicic acid, aluminum hydroxide, calcium silicates, and polyamide powder, or mixtures of these substances. Sprays can additionally contain customary propellants, such as chlorofluorohydrocarbons and volatile unsubstituted hydrocarbons, such as butane and propane.

The inhibitor(s) can be alternatively administered by aerosol. This is accomplished by preparing an aqueous aerosol, liposomal preparation, or solid particles containing the compound. A nonaqueous (e.g., fluorocarbon propellant) suspension could be used. Sonic nebulizers are preferred because they minimize exposing the agent to shear, which can result in degradation of the compound.

Ordinarily, an aqueous aerosol is made by formulating an aqueous solution or suspension of the agent together with conventional pharmaceutically acceptable carriers and stabilizers. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars, or sugar alcohols. Aerosols generally are prepared from isotonic solutions.

Transdermal patches have the added advantage of providing controlled delivery of an inhibitor(s) to the body. Such dosage forms can be made by dissolving or dispersing the agent in the proper medium. Absorption enhancers can also be used to increase the flux of the inhibitor(s) across the skin. The rate of such flux can be controlled by either providing a rate controlling membrane or dispersing the inhibitor in a polymer matrix or gel.

Pharmaceutical compositions of this invention suitable for parenteral administration comprise one or more inhibitors(s) in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents, and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Injectable depot forms are made by forming microencapsule matrices of inhibitor(s) in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the inhibitors(s) of the present invention are administered as pharmaceuticals to humans and animals, they can be given per se or as a pharmaceutical composition containing, for example, 0.1 to 99.5% (more preferably, 0.5 to 90%) of active ingredient in combination with a pharmaceutically acceptable carrier.

The preparations of agents may be given orally, parenterally, topically, or rectally. They are of course given by forms suitable for each administration route. For example, they are administered in tablets or capsule form, by injection, inhalation, eye lotion, ointment, suppository, infusion, topically by lotion or ointment, and rectally by suppositories. Oral administration is preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection, and infusion.

The phrases “systemic administration,” “administered systemically,” “peripheral administration” and “administered peripherally” as used herein mean the administration of a ligand, drug, or other material other than directly into the central nervous system, such that it enters the patient's system and thus, is subject to metabolism and other like processes, for example, subcutaneous administration.

These inhibitors(s) may be administered to humans and other animals for therapy by any suitable route of administration, including orally, nasally, as by, for example, a spray, rectally, intravaginally, parenterally, intracisternally, and topically, as by powders, ointments or drops, including buccally and sublingually.

Regardless of the route of administration selected, the inhibitor(s), which may be used in a suitable hydrated form, and/or the pharmaceutical compositions of the present invention, are formulated into pharmaceutically acceptable dosage forms by conventional methods known to those of skill in the art.

The concentration of a disclosed compound in a pharmaceutically acceptable mixture will vary depending on several factors, including the dosage of the compound to be administered, the pharmacokinetic characteristics of the compound(s) employed, and the route of administration. In general, the compounds of this invention may be provided in an aqueous physiological buffer solution containing about 0.1-10% w/v of compound for parenteral administration. Typical dose ranges are from about 0.01 to about 50 mg/kg of body weight per day, given in 1-4 divided doses. Each divided dose may contain the same or different compounds of the invention. The dosage will be an effective amount depending on several factors including the overall health of a patient, and the formulation and route of administration of the selected compound(s).

The compounds of the invention may be administered to an animal alone or in combination with pharmaceutically acceptable carriers, as noted above, the proportion of which is determined by the solubility and chemical nature of the compound, chosen route of administration and standard pharmaceutical practice.

The dosage of the compounds and/or compositions of the invention can vary depending on many factors such as the pharmacodynamic properties of the compound, the mode of administration, the age, health and weight of the recipient, the nature and extent of the symptoms, the frequency of the treatment and the type of concurrent treatment, if any, and the clearance rate of the compound in the animal to be treated. One of skill in the art can determine the appropriate dosage based on the above factors. The compounds of the invention may be administered initially in a suitable dosage that may be adjusted as required, depending on the clinical response. As an example, the compounds of the invention can be administered in a range from about 1 nanomolar to about 100 micromolar, preferably 50 nanomolar to 50 micromolar. For ex vivo treatment of cells over a short period, for example for 30 minutes to 1 hour or longer, higher doses of compound may be used than for long term in vivo therapy; for example, concentrations of 50 μM or higher may be used.

### EXAMPLES

### Example 1

### Materials and Methods

Test Compound Preparation

Each test compound was weighed between the ranges of 0.5-2.0 mg, and then dissolved in a specified volume of DMSO to produce a solution of 10 mM. Appropriate dilutions in cell culture medium were made to produce a stock solution of 400 μM. All subsequent dilutions were also done in cell culture medium. This stock solution was then used for further dilutions to produce the initial concentration to be added to the 50 μL and 450 μL of cells in the 96-well and the 48-well microtiter plates, respectively:

96-Well Microtiterplates

The initial concentration for cells that were cultured in 50 μL 96-well plates was 40 μM. Once 50 μL of this test solution was added to the cells, this then produced a final dilution of 1:2, giving the desired concentration of 20 μM. Further 1:2 dilutions were made until the desired concentration of 0.156 nM was reached.

Compound 1 was used as positive control at concentrations of 1 μM and 3 μM. Cells were incubated with the test compounds for 72 hours. C1 and OCI-AML2 and RPMI8226 cell culture media were assayed by addition of 20 μL of Alamar Blue reagent and incubated for 4 hours before reading. Cell counting was utilised for K562 and DAUDI. 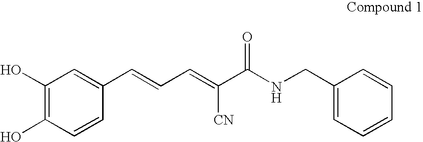
Compound Administration, Schedule and Regimen

All cell lines were cultured in RPMI 1640 cell culture medium containing 10% FCS. The cell culture medium was supplemented with penicillin-streptomycin (50 IU/mL, 50 μg/mL final concentration). All cells were cultured between the threshold concentrations (Table 1). Seeding concentrations for each cell line is provided in Table 1:

TABLE 1
Relevant information for each cell line

| Cell Line | Seeding Concentration (cells per well × 10⁵) |
|---|---|
| OCI-AML2 | 1.5 |
| C1 | 3.4 |
| RS4-11 | 0.3 |
| MOLT-4 | 0.64 |
| MV4-11 | 0.64 |
| CCRF-CEM | 0.64 |
| MOLM-13 | 0.64 |
| KAUMI-1 | 0.3 |
| SKM-1 | 0.3 |
| PEER | 0.5 |
| SU P-B15 | 0.8 |

50 μL of cell culture medium containing the test compound was added to each well twenty four hours after seeding. The final DMSO concentration in the incubation mixture was less than 0.05% (v/v) for the highest concentration test article and respectively lower for the lower concentration test articles. Proliferation of cells was assessed using the fluorimetric Alamar Blue (CellTiter Blue) test and cell counting.

Data Acquisition

Either the Alamar Blue assay or the WST-1 assay was used to determine the anti-proliferative activity of the pools containing the test compounds.

For the Alamar Blue assay, untreated cells were used as growth controls. Data were assessed in duplicates. For each compound 8 dilutions in steps of 1:2 starting with 20 μM and ending with 0.16 μM were done. The Alamar Blue cell viability assay was used to determine the IC₅₀ concentrations for the inhibition of cell proliferation or induction of cell death by the ets articles as well as the reference Compound A. Primary data from all assays were plotted for each test compound, cell system and test concentration, with the y-axis representing the readout for cell proliferation/viability and the x-axis representing the compound concentration (μM). The area of the graph representing values around 50% of the maximum control (uninhibited growth) value was then expanded at least ten-fold in order to accurately ascertain the IC₅₀ values (in μM) for each compound. This semi-manual method was compared previously with the various software-based solutions for IC₅₀ calculations and found to be superior.

Alamar Blue Assay

The Alamar Blue-based CellTiter-Blue assay indicates cell viability by measuring the ability of cells in culture to reduce resazurin to resorufin, whereby the intensity of the fluorescence signal is directly proportional to the number of live cells and hence an indirect indicator of cell proliferation.

Based on preliminary experiments, cultures of all cell lines were seeded at the appropriate seeding concentrations in 50 μL/well (Table 1). After 24 hrs the cells were incubated for 72 hours with the test compound. Following addition of CellTiter-Blue Reagent (20 μL/well) and brief mixing, cells were incubated for another 4 hours before fluorescence was measured (Ex/Em of 560/590 nm) using a Spectramax Gemini XPS microplate reader (Molecular Devices, Surrey Hills, VIC, Australia).

WST-1 Assay

The WST-1 cell proliferation assay relies on the formation of a formazan from a tetrazolium salt by cellular enzymes, whereby the amount of the obtained formazan dye as an indicator of viability is directly proportional to the number of metabolically active cells in culture.

Following 72 hours incubation of the respective cells in 50 μL/well with test compound, 10 μL of WST-1 reagent was added to each well and its contents briefly mixed. The cells were then incubated for 2 hours before absorbance of the dye was measured at 450 nm using a commercial UV/VIS microplate reader (Molecular Devices).

### Example 2

### Cellular Activity for Compound A

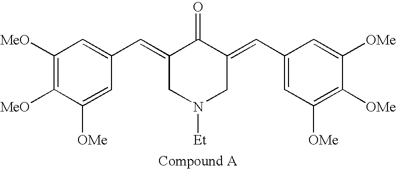 Human Leukemia Cell Lines

| | Compound A |
|---|---|
| Cell Line | [IC₅₀] μM |
| OCI-AML2 (AML)¹⁾ | 1.03 |
| MOLM-13 (AML)¹⁾ | 1.90 |
| KASUMI-1 (AML)¹⁾ | 0.56 |
| SKM-1 (AML)¹⁾ | 0.56 |
| MV4-11 (AML)¹⁾ | 0.22 |
| C1 (T-ALL)¹⁾ | 0.18 |
| CCRF-CEM (T-ALL)¹⁾ | 1.18 |
| MOLT-4 (T-ALL)¹⁾ | 0.97 |
| PEER (B-ALL)¹⁾ | 0.72 |
| SUP-B15 (B-ALL)¹⁾ | 1.03 |
| RS4-11 (B-ALL)¹⁾ | 0.45 |
| K562 (CML)²⁾ | 2.50 |
| RPMI8226 (myeloma)¹⁾ | 1.50 |
| DAUDI (non-Hodgkin | 0.55 |
| lymphoma)²⁾ | |
| OCI-AML2 (AML)¹⁾ | 1.03 |
| MOLM-13 (AML)¹⁾ | 1.90 |
| KASUMI-1 (AML)¹⁾ | 0.56 |
| SKM-1 (AML)¹⁾ | 0.56 |
| MV4-11 (AML)¹⁾ | 0.22 |
| C1 (T-ALL)¹⁾ | 0.18 |
| CCRF-CEM (T-ALL)¹⁾ | 1.18 |
| MOLT-4 (T-ALL)¹⁾ | 0.97 |
| PEER (B-ALL)¹⁾ | 0.72 |
| SUP-B15 (B-ALL)¹⁾ | 1.03 |
| RS4-11 (B-ALL)¹⁾ | 0.45 |
| K562 (CML)²⁾ | 2.50 |
| RPMI8226 (myeloma)¹⁾ | 1.50 |
| DAUDI (non-Hodgkin | 0.55 |
| lymphoma)²⁾ | |

¹⁾Alamar Blue Assay
²⁾Trypan Blue Dye Exclusion and Cell Counting Assay

### Example 3

### Oral Mouse Pharmacokinetic Activity for Compound A

Compound A (Stored at ca. +5° C.)

Formulation preparation for intravenous route:

| | | |
|---|---|---|
| Mass for preparation: | 5.11 | mg |

| | |
|---|---|
| Notional purity: | 100% |

Composition of excipient/volumes:

| | | | |
|---|---|---|---|
| Absolute ethanol: | 0.8 | mL | |
| PEG300: | 3.2 | mL | |
| Total volume of excipients: | 4 | mL | +ultrasonic bath 5 minutes |
| Concentration: | 1.28 | mg/mL | |
| Administered volume: | 3 | mL/kg | |
| Administered dose: | 3.83 | mg/kg | |

Formulation preparation for oral routes:

| | | |
|---|---|---|
| Mass for preparation: | 50.15 | mg |

| | |
|---|---|
| Notional purity: | 100% |

Composition of excipient/volumes:

| | | | |
|---|---|---|---|
| Carboxymethylcellulose 1%/Tween80 0.2% in | 10 | mL | +ultrasonic bath 10 minutes |
| water: | | | |
| Concentration: | 5.02 | mg/mL | and permanent magnetic stirring |
| Administered volume for oral at low dose: | 2 | mL/kg | (suspension) |
| Administered dose for oral route at low dose: | 10.0 | mg/kg | |
| Administered volume for oral at high dose: | 10 | mL/kg | |
| Administered dose for oral route at high dose: | 50.2 | mg/kg | |

Animal Phase:
Species: Mice CD1 (supplied from Harlan, France)
Routes tested: Intravenous (Mice Nos.39 to 52 incl.), oral low dose (Mice Nos.53 to 63 incl.) and oral high dose (Mice Nos.65 to 76 incl.)
   Animals were not fasted.

Times of blood sampling performed from the abdominal aorta (2 animals per time point):
Intravenous route (IV): 5 min, 15 min, 30 min, 1 h, 2 h, 6 h and 24 h
Oral routes (PO): 15 min, 30 min, 1 h, 3 h, 6 h and 24 h

Plasma was harvested by centrifugation of blood at 3500 g for 10 minutes at ca. +5° C.

Plasma was stored at ca. −20° C. until analysis.

Analytical Phase:

Analytical method: LC/MSMS after extraction

Calibration undertaken in plasma (5 levels in mice, twice injected, ranging from 10 to 1000 ng.mL⁻¹)

Description of the Analytical Method:

HPLC Conditions:
Colonne: Zorbax C18 50×2.1 mm, 3.5 μm
Filter: A102X 5 μm
Injected volume: 20 μL
Mobile phase flow: 0.1 mL.min⁻¹
Autosampler temperature: ca. +5° C.
Oven temperature: ca. +30° C.
Run time: 10 min
Mobile phase: deionized water with 0.1% formic acid/methanol with 0.1% formic acid; 30:70 v/v (isocratic)
Retention time: ca. 2.1 min

Mass Spectrometer Conditions:

| Ionisation | | | | |
|---|---|---|---|---|
| mode | Transition | Dwell (s) | Cone (V) | Ecoll (eV) |
| ESI+ | 484.13 > 181.33 | 0.3 | 42 | 31 |

Calibration Standards:
Stock solutions of compound were prepared in DMSO

Spiking solutions were prepared in methanol according to the table below:

| Concentration of | |
|---|---|
| spiking solution | Concentration in |
| (ng/mL) | plasma (ng/mL) |
| 500 | 1000 |
| 200 | 400 |
| 50 | 100 |
| 10 | 20 |
| 5 | 10 |

Extraction Method for Plasma Sample:
50 μL of plasma combined with 100 μL of methanol
Vortexed a few seconds
Centrifuged 3 min at 13 000 rpm at ca. +5° C.
Diluted 50 μL of the supernatant with 50 μL of deionized water in a HPLC vial for injection
Vortexed a few seconds
Injected into the HPLC/MSMS system
   Extraction Method for Plasma Calibration Sample:
50 μL of plasma sample combined with 100 μL of spiking solution
Vortexed a few seconds
Centrifuged 3 min at 13 000 rpm at ca. +5° C.
Diluted 50 μL of the supernatant with 50μL of deionized water in a HPLC vial for injection
Vortexed a few seconds
Injected into the HPLC/MSMS system
   Results:
   Animal and Analytical Phase Results:

Intravenous Route:

| | | | Dose | |
|---|---|---|---|---|
| Time | Mouse | Bodyweight | volume | Plasma levels |
| (h) | ID. | (g) | (mL) | (ng · mL⁻¹) |
| 0.083 | 39 | 31 | 0.09 | 199.57 |
| | 40 | 31 | 0.09 | 333.91 |
| 0.25 | 41 | 32 | 0.10 | 64.39 |
| | 42 | 31 | 0.09 | 85.28 |
| 0.5 | 43 | 29 | 0.09 | 57.52 |
| | 44 | 30 | 0.09 | 69.56 |
| 1 | 45 | 30 | 0.09 | 80.73 |
| | 46 | 32 | 0.10 | 39.25 |
| 2 | 47 | 32 | 0.10 | 17.9 |
| | 48 | 29 | 0.09 | 12.9 |
| 6 | 49 | 34 | 0.10 | 14.94 |
| | 50 | 32 | 0.10 | 7.29 |
| 24 | 51 | 28 | 0.06 | 1.68 |
| | 52 | 33 | 0.10 | 0.36 |

Italics: <LOQ (10 ng · mL⁻¹)

Oral Route at Low Dose:

| | | | Dose | |
|---|---|---|---|---|
| Time | Mouse | Bodyweight | volume | Plasma levels |
| (h) | ID. | (g) | (mL) | (ng · mL⁻¹) |
| 0.25 | 53 | 32 | 0.06 | 67.54 |
| | 54 | 31 | 0.06 | 178.37 |
| 0.5 | 55 | 30 | 0.06 | 71.71 |
| | 56 | 32 | 0.06 | 83.39 |
| 1 | 57 | 30 | 0.06 | 83.51 |
| | 58 | 32 | 0.06 | 70.35 |
| 3 | 59 | 35 | 0.07 | 41.7 |
| | 60 | 33 | 0.07 | 40.47 |
| 6 | 61 | 34 | 0.07 | 2.67 |
| | 62 | 30 | 0.06 | 6.30 |
| 24 | 63 | 30 | 0.06 | 5.80 |
| | 64 | 33 | 0.07 | ND |

Italics: <LOQ (10 ng · mL⁻¹);
ND: Not Detectable

Oral Route at High Dose

| | | | Dose | |
|---|---|---|---|---|
| Time | Mouse | Bodyweight | volume | Plasma levels |
| (h) | ID. | (g) | (mL) | (ng · mL⁻¹) |
| 0.25 | 65 | 34 | 0.34 | 818.79 |
| | 66 | 34 | 0.34 | 444.58 |
| 0.5 | 67 | 30 | 0.30 | 433.97 |
| | 68 | 34 | 0.34 | 393.99 |
| 1 | 69 | 28 | 0.28 | 175.49 |
| | 70 | 32 | 0.32 | 213.68 |
| 3 | 71 | 36 | 0.36 | 137.48 |
| | 72 | 31 | 0.31 | 160.65 |
| 6 | 73 | 30 | 0.30 | 23.07 |
| | 74 | 26 | 0.26 | 137.34 |
| 24 | 75 | 37 | 0.37 | 2.36 |
| | 76 | 32 | 0.32 | 1.04 |

Italics: <LOQ (10 ng · mL⁻¹)

Pharmacokinetic Analysis:

Intravenous Route:

| Parameter | Value |
|---|---|
| AUClast | 245.9 |
| (ng/mL * h) | |
| AUCtot | 254.1 |
| (ng/mL * h) | |
| % AUCextra | 3.2 |
| T½ (h) | 5.5 |
| Total Clearance | 15071 |
| (mL/h/kg) | |
| Vd (mL/kg) | 119389 |

Oral Route at Low Dose:

| Parameter | Value |
|---|---|
| Cmax (ng/mL) | 123.0 |
| Tmax(h) | 0.25 |
| AUClast | 307.9 |
| (ng/mL * h) | |
| AUCtot | 321.1 |
| (ng/mL * h) | |
| % AUCextra | 4.1 |
| Bioavailability | 48.0 |
| factor (%) | |

ND: Not determined

Oral Route at High Dose:

| Parameter | Value |
|---|---|
| Cmax (ng/mL) | 631.7 |
| Tmax(h) | 0.25 |
| AUClast | 1394.7 |
| (ng/mL * h) | |
| AUCtot | 1402.6 |
| (ng/mL * h) | |
| % AUCextra | 0.57 |
| Bioavailability | 43.4 |
| factor (%) | |

Equivalents

Those skilled in the art will recognize, or be able to ascertain using no more than routine experimentation, numerous equivalents to the compounds and methods of use thereof described herein. Such equivalents are considered to be within the scope of this invention and are covered by the following claims.

All of the above-cited references and publications are hereby incorporated by reference.

We claim:

1. A pharmaceutical composition, comprising a compound of Formula Ia, or a salt, solvate, or hydrate thereof and a pharmaceutically acceptable carrier: 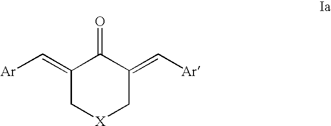
wherein
Ar and Ar′ are independently a phenyl ring substituted one to three times with substituents selected from alkyl, haloalkyl (up to perhalo), alkoxy, and haloalkoxy (up to perhalo);
X is NR; and
R is selected from H and alkyl.

2. A pharmaceutical composition of claim 1, wherein R is C₁₋₆alkyl.

3. A pharmaceutical composition of claim 2, wherein R is selected from methyl and ethyl.

4. A pharmaceutical composition of claim 3, wherein R is ethyl.

5. A pharmaceutical composition of claim 3, wherein Ar and Ar′ both bear a substituent at the 3-position relative to the point of attachment to the rest of the molecule.

6. A pharmaceutical composition of claim 5, wherein both Ar and Ar′ are substituted at the 3-, 4-, and 5-positions relative to the point of attachment to the rest of the molecule and the substituents are independently selected from alkyl and alkoxy.

7. A pharmaceutical composition of claim 6, wherein the substituents are all methoxy.

8. A pharmaceutical composition of claim 1, wherein the compound of formula Ia is compound A

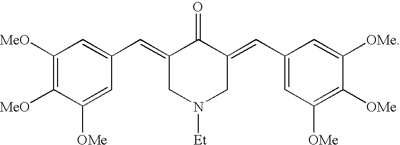

9. A pharmaceutical composition, comprising a compound of Formula I, or a salt, solvate, or hydrate thereof and a pharmaceutically acceptable carrier: 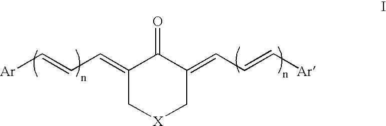
wherein
Ar and Ar′ are independently a phenyl ring optionally substituted with one or more substituents selected from halo, cyano, sulfate, phosphate, SO₃H, P(═O)(OH)₂, NO₂, alkyl, haloalkyl (up to perhalo), OH, alkoxy, CH₃O(CH₂CH₂O)_{q}CO₂—, haloalkoxy (up to perhalo), —O—Si(alkyl)(alkyl)(alkyl), HO₂C—, alkylO₂C—, alkylC(O)—, alkylCO₂—, alkylOCO₂—, (R⁶)(R⁷)N—, R⁷C(O)N(R⁶)—, (R⁶)(R⁷)NCO—, (R⁶)(R⁷)NC(O)N(R⁶), (R⁶)(R⁷)NCO₂—, SH, alkylthio, and —O(CH₂)_{y}O—;
each y is, independently for each occurrence, an integer from 1 to 6, thereby forming a ring fused to the phenyl ring;
R⁶ and R⁷ are each independently selected from hydrogen and C₁₋₆alkyl, or R⁶ and R⁷ together are —(CH₂)ₚY(CH₂)ₚ—, thereby forming a ring;
each p is, independently for each occurrence, an integer from 1 to 3;
Y is selected from O, S, NH, NC₁₋₆alkyl, and CH₂;
each n is, independently for each occurrence, either 0 or 1;
X is selected from O, NR, and (CH₂)ₘ;
R is selected from H, OH, and alkyl; and
m is an integer from 0 to 3;
provided that when Ar and Ar′ are both substituted at each of the 3-, 4-, and 5-positions, relative to the point of attachment to the rest of the molecule, and the 3- and 4-positions are each substituted with OCH₃, then the substituent at the 4-position is not OH.

10. A method for the treatment of cancer, comprising administering a compound of Formula Ia, or a salt, solvate, or hydrate thereof: 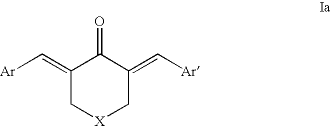
wherein
Ar and Ar′ are independently a phenyl ring substituted one to three times with substituents selected from alkyl, haloalkyl (up to perhalo, e.g., perfluoro), alkoxy, and haloalkoxy (up to perhalo, e.g., perfluoro);
X is NR; and
R is selected from H and alkyl.

11. A method of claim 10, wherein R is C₁₋₆alkyl.

12. A method of claim 11, wherein R is selected from methyl and ethyl.

13. A method of claim 12, wherein R is ethyl.

14. A method of claim 10, wherein Ar and Ar′ both bear a substituent at the 3-position relative to the point of attachment to the rest of the molecule.

15. A method of claim 14, wherein both Ar and Ar′ are substituted at the 3-, 4-, and 5-positions relative to the point of attachment to the rest of the molecule and the substituents are independently selected from alkyl and alkoxy.

16. A method of claim 10, wherein the compound of formula Ia is compound A

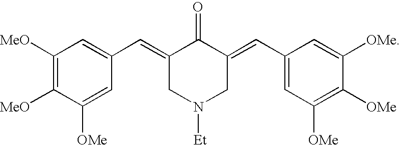

17. A method for the treatment of cancer, comprising administering a compound of Formula I, or a salt, solvate, or hydrate thereof: 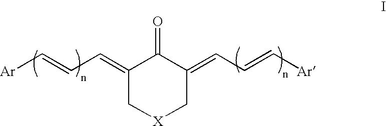
wherein
Ar and Ar′ are independently a phenyl ring optionally substituted with one or more substituents selected from halo, cyano, sulfate, phosphate, SO₃H, P(═O)(OH)₂, NO₂, alkyl, haloalkyl (up to perhalo), OH, alkoxy, CH₃O(CH₂CH₂O)_{q}CO₂—, haloalkoxy (up to perhalo), —O—Si(alkyl)(alkyl)(alkyl), HO₂C—, alkylO₂C—, alkylC(O)—, alkylCO₂—, alkylOCO₂—, (R⁶)(R⁷)N—, R⁷C(O)N(R⁶)—, (R⁶)(R⁷)NCO—, (R⁶)(R⁷)NC(O)N(R⁶), (R⁶)(R⁷)NCO₂—, SH, alkylthio, and —O(CH₂)_{y}O—;
each y is, independently for each occurrence, an integer from 1 to 6, thereby forming a ring fused to the phenyl ring;
R⁶ and R⁷ are each independently selected from hydrogen and C₁₋₆alkyl, or R⁶ and R⁷ together are —(CH₂)ₚY(CH₂)ₚ—, thereby forming a ring;
each p is, independently for each occurrence, an integer from 1 to 3;
Y is selected from O, S, NH, NC₁₋₆alkyl, and CH₂;
each n is, independently for each occurrence, either 0 or 1;
X is selected from O, NR, and (CH₂)ₘ;
R is selected from H, OH, and alkyl; and
m is an integer from 0 to 3.

18. A method of claim 10, wherein the cancer is selected from leukemia, a lymphoma, a myeloma, a carcinoma, or a hematopoietic cell cancer.

19. A method of claim 18, wherein the cancer is a hematopoietic cell cancer.

20. A method of claim 18, wherein the cancer is a leukemia selected from acute lymphoblastic leukemia, Philadelphia+leukemia, Philadelphia−leukemia, acute myelocytic leukemia, chronic myeloid leukemia, chronic lymphocytic leukemia, and juvenile myelomonocyte leukemia.

21. A method of claim 20, wherein the leukemia is acute lymphoblastic leukemia.
